(19) United States
(12) Patent Application Publication
(10) Pub. No.: US 2004/0219618 A1
(43) Pub. Date: Nov. 4, 2004

(54) Identifying micro-organisms

(76) Inventors: Richard William Titball, Wiltshire (GB); Dominique Despeyroux, Wiltshire (GB)

Correspondence Address: JOHN S. PRATT, ESQ, KILPATRICK STOCKTON, LLP, 1100 PEACHTREE STREET, ATLANTA, GA, 30309, US

(21) Appl. No.: 10/478,981

(22) PCT Filed: May 22, 2002
(86) PCT No.: PCT/GB02/02208

(30) Foreign Application Priority Data
May 22, 2001 (GB) 0112428.8

(51) Int. Cl. G01N 33/554; G01N 33/569; C12Q 1/04

(52) U.S. Cl. 435/7.32

(57) ABSTRACT

A method of rapidly identifying unknown micro-organisms by means of mass spectrometry of biomarkers that are isolated from lysates of the micro-organisms on the basis of their structural similarity across a number of species. Also disclosed are said biomarkers, in particular Hsp60.

### FIELD OF THE INVENTION

The invention relates to the rapid identification of micro-organisms, such as bacteria.

### BACKGROUND

There are many situations in which it is desirable to be able to identify potentially pathogenic organisms such as bacteria and viruses rapidly. Current laboratory methods typically involve culturing organisms and the use of immunodiagnostic tests, or preparation of histological specimens and use of specialised staining and/or immunohistochemical techniques. Such techniques demand, at best, hours and, where culturing of organisms is required, days. DNA-based identification, for instance PCR, although more sensitive, still requires hours, as well demanding considerable laboratory facilities and expertise.

The identification of specific micro-organism by means of specific polyclonal antisera or of monoclonal antibodies is standard practice. Immunohistochemistry allows identification of organisms present in tissues, and techniques such as enzyme-linked immunosorbent assays (ELISA) are used to detect pathogens, or antigens derived from them, in body fluids. However, unless being used only to confirm the presence of a particular pathogen suggested by other diagnostic criteria (the usual situation), these diagnostic techniques require the use of a battery of different specific antibodies and are poorly suited to the identification of a particular pathogen from a large number of potential targets. Immunoaffinity purification of cellular components is also well-known in the art, but is not generally a useful technique for identification purposes, since it usually requires knowledge of the organism concerned.

The use of cross-reacting sera to identify micro-organisms has been previously reported (Bonenberger et al, 2001). In this case, polyclonal antiserum raised against the BCG strain of Mycobacterium bovis was used to stain a wide variety of micro-organisms in biopsy specimens. This technique did not allow identification of specific individual organisms, but generically stained a range of bacterial, fungal and protozoal pathogens.

Mass spectrometry (MS) has been used increasingly for biological applications in recent years. New developments have allowed large biological molecules to be analysed (reviewed in Bakhtier and Tse, 2000). In particular, matrix-assisted desorption ionisation (MALDI) and electrospray MS with their relatively gentle ionisation methods are particularly well-suited to protein applications (reviewed in Rowley et al, 2000). More recently, the introduction of ion trap MS has reduced the time required to analyse mixtures of biological molecules, particularly when small amounts are available (Henderson et al, 1999).

WO 00/29987 (Demirev/University of Maryland) discloses a method of measuring the molecular masses of various components of micro-organisms and using database searching to attempt to identify them. However, such an approach has the disadvantages of having to process large amounts of information and of having to distinguish between a great number of components of similar molecular mass. There is no attempt to simplify the mass spectrometry by any preselection of informative biomarkers.

WO 96/37777 (Nelson et al) discloses a method for analysing antibody/antigen analytes using mass spectrometry. However, the object of the application is to determine the presence or absence of specific antibodies and/or antigens, and, if so, to measure the amounts present. There is no suggestion of a method that might be used to unambiguously identify an unknown organism.

In combination with the use of specific monoclonal antibodies and immunoaffinity purification, MS has allowed detailed structural mapping of many molecules

(reviewed in Downard, 2000). In particular, the analysis of molecules of interest by immunoaffinity chromatography, followed by MS analysis of the isolated molecules has been performed on many proteins, for example calnexin (Yamashita et al, 1999). In some cases, proteins purified by immunoaffinity are then subjected to enzymatic digestion to generate a set of defined peptides, which are then analysed by MS, for example the Ty1 Gag protein of Saccharomyces cerevisiae (Yu et al, 1998). Lacey et al (2001) report the analysis of isoforms of transferrin by means of immunoaffinity purification followed by MS analysis in order to establish the structure of the carbohydrate modifications responsible for the heterogeneity of transferrin. However, this involved the use of specific anti-transferrin polyclonal antibodies binding to molecules of the same amino acid sequence. The differences between molecules were within carbohydrate part of the glycoproteins and the antibodies were not cross-reactive.

Affinity purification of molecules carrying a common structural feature may be performed with other ligands than immobilised antibodies and is a technique very well-known in the art. Bundy and Fenselau (2001) report the use both of lectins to capture a variety of complex carbohydrates from a variety of micro-organisms, and of defined carbohydrates to capture bacteria expressing lectin molecules. The captured molecules, or peptides derived from them by acid hydrolysis, were then analysed by MS. Although these could be described as generic ligands used to capture a variety of molecules for subsequent MS analysis, the method is not used for the identification of unknown organisms. There is no selection of suitable biomarkers for such an application. There is no teaching as to how such biomarkers might be identified. There is no suggestion that the ligands suggested would have sufficient specificity to allow purification of the specific biomarker required or that the biomarkers used would be consistently present across the necessary variety of organisms required for the present invention. There is no suggestion that the use of one, or a small set, of biomarkers might allow rapid identification of a wide range of micro-organisms.

Although more rapid identification of pathogens in a laboratory setting would be advantageous in itself, there is a further need for portable field-based systems that could be employed in both peacetime epidemics of human or animal disease, and in a military situation as part of biological weapons counter-measures. In this context, pathogens, such as plague or anthrax bacteria, may be used by an aggressor, typically delivered as an aerosol. Laser measurements may be used to detect the presence of an aerosol , but this may simply be a mist of, say, water, delivered as a dummy weapon (Willeke and Baron, 1993). There is a need to be able rapidly to make an accurate identification, in the field, of matter, however delivered, which is suspected to be a biological weapon. Ion trap MS-based approaches to identification of bacteria have been reported previously (Krishnamurthy et al, 1999). In this case, following separation by reverse-phase microcapillary chromatography, whole bacteria were directly analysed, and identified purely on the basis of the spectrum produced. Although the authors comment on the potential for miniaturisation of the equipment for field use, there is no suggestion of the use of any form of immunoaffinity selection to simplify the MS analysis required and to make such analysis practical for rapid identification of a large range of organisms. In addition, there is no reference to the identification of particular proteins for use as biomarkers, and no consideration of the reproducibility the MS spectrum obtained in different environmental conditions. Without characterisation of the biomarkers used, this technique is also vulnerable to inconsistency in the behaviour of the biomarkers in, for instance, ionisation characteristics, which further reduces its reliability.

Direct MS analysis of viral proteins has also been reported (WO 99/58727), but again, no affinity purification or use of common biomarkers is suggested. MS analysis of bacterial cell lysates has also been reported (Chong et al, 1997). Bacterial samples were solubilised with guanidinium hydrochloride and Triton X-100 before analysis by MALDI-TOF MS. There was no use of any form of affinity purification and the aim was to profile induction and repression of protein synthesis in Escherchia coli, rather than to identify unknown organisms.

### STATEMENT OF INVENTION

The invention provides a method of identifying a micro-organism comprising determining the molecular mass of at least one protein extracted from the plurality of proteins which constitute the micro-organism.

The invention follows from the discovery that, of all the thousands of proteins which typically constitute a micro-organism, an identification can be made by assessing a relatively very small selection of proteins, even as few as one. It is well-known in the art that a number of proteins, often those that perform some ubiquitous and vital metabolic function within the cell, are highly structurally conserved across a broad range of species. The fact that they perform very similar functions in different species sharing common metabolic pathways results in evolutionary pressure to conserve structural features on which functional properties depend. Such highly conserved proteins include enzymes concerned with basic cellular processes like glycolysis (eg triose phosphate isomerase) and nucleotide metabolism (eg adenylate kinase), DNA polymerases and heat shock proteins.

Regions of such proteins that are conserved show a high degree of homology in amino acid sequence. As a result, they bear common immunological epitopes to which cross-reacting antibodies may bind so that a single monoclonal antibody may used to identify, or to isolate, of any of a family of such conserved proteins from a variety of species. In some cases a single antibody may bind to such a very widely conserved epitope and so be useful in isolating proteins from many species. In many cases, however, a number of such antibodies, binding to different epitopes on the same, or other proteins, may be used in combination, in order to maximise the number of species identifiable and minimise the chance of a micro-organism that is present remaining undetected. Surprisingly, despite their highly conserved structure, the current invention demonstrates that the small differences between such proteins allow rapid and consistent identification of the species from which they are derived by accurate determination of their mass. The resolution obtained from mass spectrometry is easily capable of identifying single amino acid differences between proteins or peptides derived from them. Thus, the combination of affinity purification of highly conserved proteins bearing common epitopes, and subsequent mass spectroscopic analysis of such proteins, or peptides derived from them, may form the basis of a rapid and reliable method of identifying the micro-organism from which they are obtained. Proteins, or other biological molecules, used in this way are known as biomarkers.

This method depends on the availability of a database of biomarkers, relating accurate molecular masses of known biomarkers to the species from which they are derived. In some cases, it may be necessary to use more than one biomarker for unambiguous identification of a species, sub-species or strain. Such databases are generated by growing the relevant micro-organisms under a range of conditions, mapping the proteomes by 2D-gel electrophoresis and with western blot using antibodies raised against the whole micro-organism cell lysate. Markers of interest, selected according to the criteria below, can rapidly be identified, their masses accurately determined by mass spectroscopy and the mass entered into the database.

An important factor in the selection of biomarkers is that that their masses should be constant irrespective of variable factors such as cell cycle, or of growth conditions such as temperature or availability of nutrients. This is particularly relevant to the identification of micro-organisms in the environment, such as biological weapons, where conditions may be far from optimal for the organism concerned, and in response to which stress it may change its pattern of gene expression or of post-translational modification. It is therefore preferable that they are not transiently modified by phosphorylation, lipidation or ribosylation, although if such modifications were known and consistent, this would not preclude the use of such molecules for identification. Biomarkers should also be consistently expressed, in all conditions, at levels high enough to make extraction and isolation in quantities large enough to make identification practical.

In view of such considerations, the heat shock protein (Hsp) families of molecules are particularly suitable biomarkers. Molecules such Hsp60 are highly conserved across species, are not post-translationally modified, and are consistently and ubiquitously expressed. In fact, their expression, since it is related to cellular stress, is increased when organisms are in sub-optimal environmental conditions. Hsp60 (GroEL, chaperoning, together with its co-chaperonin Hsp10 (GroES) is involved in the ATP-dependent, post-translational folding of nascent polypeptides into their correct tertiary structures, as well as refolding of non-native proteins back into their correct native conformation (Sigler et al, 1998).

In addition to the use of cross-reacting antibodies to isolate appropriate biomarkers from a range of micro-organisms, other ligands may be used for affinity capture of such biomarkers. Lectins may be used to capture glycoproteins of glycolipids carrying a specific structural feature in their carbohydrate modifications. Immobilised nucleic acids may be used to capture DNA-binding proteins. These may be generic DNA-binding proteins (such as polymerases) or may be sequence-specific binding proteins (such as transcription factors or restriction endonucleases), depending on the ligand used. Immobilised RNA aptamers and ribozymes may also be used to bind specific target structures (reviewed by Hoffman et al, 2001). Artificial dye ligands are capable of binding diverse molecules sharing a common structural feature such as a cofactor binding pocket (see Affinity Chromatography: principles and methods, Pharmacia LKB Biotechnology, 1988). As an example, Cibacron Blue F3G-A binds a variety of NAD or NADP-requiring enzymes, and enzymes that have specificity for adenylyl substrates such as adenylate kinase, which is a useful conserved biomarker for the present invention.

The combination of immunoaffinity purification of one or more highly conserved biomarkers from cell lysates using a cross-reacting antibody or some other generic binding ligand, followed by mass spectroscopic analysis of the one or more biomarkers used, preferably by ion trap mass spectroscopy, by reference to a database, provides a rapid, reliable and reproducible method of identifying micro-organisms for a variety of applications.

In an alternative embodiment, the captured biomarker may be enzymatically digested to produce a predictable set of peptides consistent with the enzyme used and the known amino acid sequence of the candidate molecules from the range of species recorded. The spectrum of masses produced is a fingerprint characteristic of the biomarker from which they originated and can be cross-referenced to a database for identification of the organism involved. The use of immobilised enzymes is a convenient way of simplifying the process for automation and also reducing the complication of enzyme molecules being present in the peptide mixture to be analysed.

For field biological warfare counter-measures applications, it is envisaged that the entire process from sampling the environment, through concentrating and lysing the cells, affinity purifying the biomarker(s) of interest, eluting said biomarkers, delivering them to the mass spectrometer, recording the mass spectrum obtained, matching the spectrum to the best fit in a database and finally cross-referencing this information to obtain an identification of the organism detected, will be automated within a portable unit. Extra steps, such as enzymatic cleavage of captured biomarkers, would be invoked automatically if a definitive identification does not result from the first analysis. A proportion of the original eluate from the affinity purification step will be retained for this purpose. Depending on the precise application, the ultimate read-out may be a precise identification of an organism or strain thereof. For battlefield applications, a simple “safe” or “not safe” read-out might be appropriate.

Further automatic units may be designed for other applications. For instance, a bench-top unit for the analysis of blood, or tissue samples for hospital and laboratory use.

Accordingly the current invention provides a biomarker characterised in that species homologues of said biomarker derived from the majority of species in at least two genera of micro-organisms are substantially structurally similar, such that said structural similarity allows isolation of said biomarkers from different species of micro-organism and that each biomarker derived from each species of micro-organism in said genera has a unique molecular mass.

Preferably, said biomarker is characterised in that it is a protein and in that said structural similarity consists of substantial similarity of amino acid sequence. It is also preferred that said micro-organisms are bacteria.

Preferably, said biomarker is characterised in that least three species homologues share at least one common epitope allowing isolation by immunoaffinity chromatography. More preferably, at least one common epitope is shared by at least five species. Even more preferably, it is a heat shock protein and, most preferably, it is Hsp60.

Alternatively, said biomarker may be adenylate kinase.

Also provided is a method of identifying micro-organisms comprising:

a. identifying a biomarker characterised in that species homologues of said biomarker derived from the majority of species in at least two genera of micro-organisms are substantially structurally similar, such that said structural similarity allows isolation of said biomarkers from different species of micro-organism and that each biomarker derived from each species of micro-organism in said genera has a unique molecular mass;

b. isolating said biomarkers by affinity chromatography chromatography directed towards regions of structural similarity;

c. measuring the mass of said biomarkers by mass spectrometry, and;

d. analysing the combination of molecular mass data obtained with reference to a database and thereby deducing the species of micro-organism present.

Preferably, said biomarkers are isolated from a cell lysate.

More preferably said biomarkers are isolated by means of immunoaffinity chromatography and, most preferably, by immobilised antibodies that bind specifically to cross-reacting epitopes present on marker molecules derived from a variety of micro-organism species.

Alternatively, the method may include the additional step of cleaving the isolated biomarkers into defined fragments before determining their molecular mass by means of mass spectroscopy. Preferably, said cleavage of biomarkers is achieved by means of enzymatic digestion.

Preferably, the measurement of molecular mass of biomarkers or fragments thereof is by means of ion trap mass spectrometry.

Also provided is a method of identifying macromolecular toxins comprising:

a. Isolating one or more toxins by affinity chromatography;

b. measuring the molecular mass of said toxin(s) by means of mass spectrometry; and

c. analysing the combination of molecular mass data obtained with reference to a database and thereby deducing the identity of the toxin(s) present.

Another embodiment of the invention comprises an apparatus for the automatic performance of any of the above comprising:

a. a means for isolating said biomarkers or toxins;

b. a unit comprising a mass spectrometer capable of determining the molecular masses of said biomarkers or toxins

c. a data processing device capable of matching the data obtained with a database of known molecular masses and thereby deducing the identity of the micro-organism or toxin detected.

Alternatively, said apparatus further comprises a unit comprising one or immobilised proteolytic enzymes capable of cleaving said biomarkers.

### Definitions

As used herein “biomarker” means an environmental biochemical parameter, detection or quantification of which may be used as a means of identifying a potential biological hazard. In this case, it specifically refers to structurally conserved biological macromolecules, including proteins, that may be isolated from a wide range of micro-organisms, and used to identify said micro-organisms.

As used herein “affinity chromatography” means “a type of adsorption chromatography in which the molecule to be purified is specifically and reversibly adsorbed by a complementary binding substance (ligand) immobilised on an insoluble support (matrix)” (see Affinity Chromatography: principles and methods, Pharmacia LKB Biotechnology, 1988).

As used herein “immunoaffinity chromatography” means a form of affinity chromatography in which the immobilised ligand is an antibody or epitope-binding derivative thereof.

As used herein “species homologue” means an equivalent gene or gene product from another species. Such homologues perform equivalent functions and share a degree of sequence similarity at the amino acid level. As used herein, no assumptions are made as to the evolutionary relationship between the organisms involved.

### DETAILED DESCRIPTION OF THE INVENTION

The invention will now be described by way of example, with reference to the figure of the drawings in which:

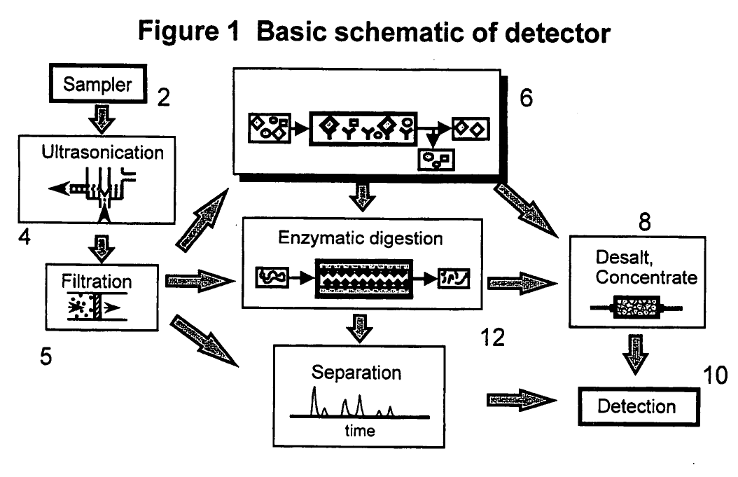

FIG. 1 is a schematic layout of the functional elements in a system utilising the method according to the invention.

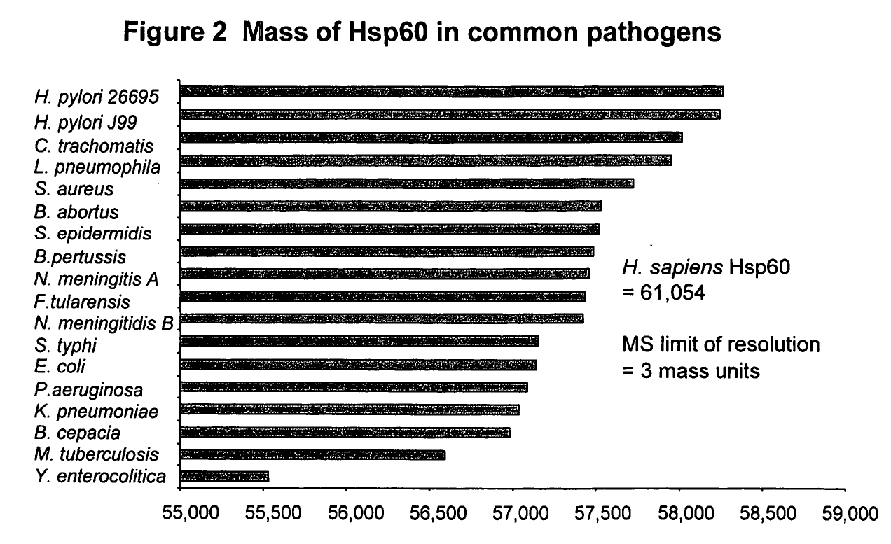

FIG. 2 shows a graphical comparison the masses of Hsp60 protein from a variety of potentially pathogenic bacteria

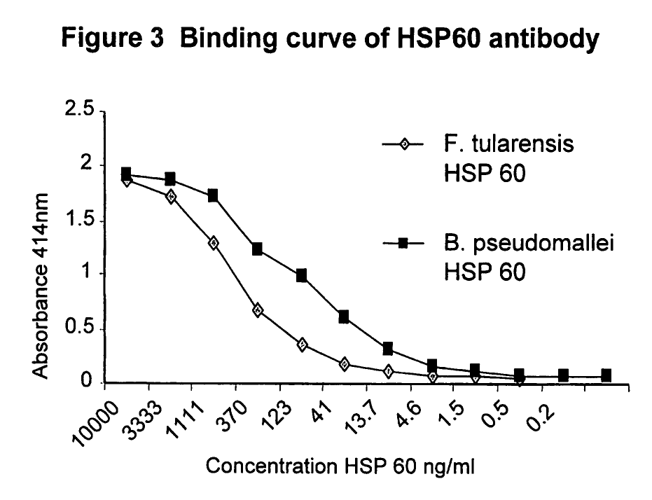

FIG. 3 shows an indirect ELISA measure of the binding affinities of monoclonal IgG₁ A57-E4 to recombinant Hsp60 proteins from Francisella tularensis and Burkholderia pseudomallei.

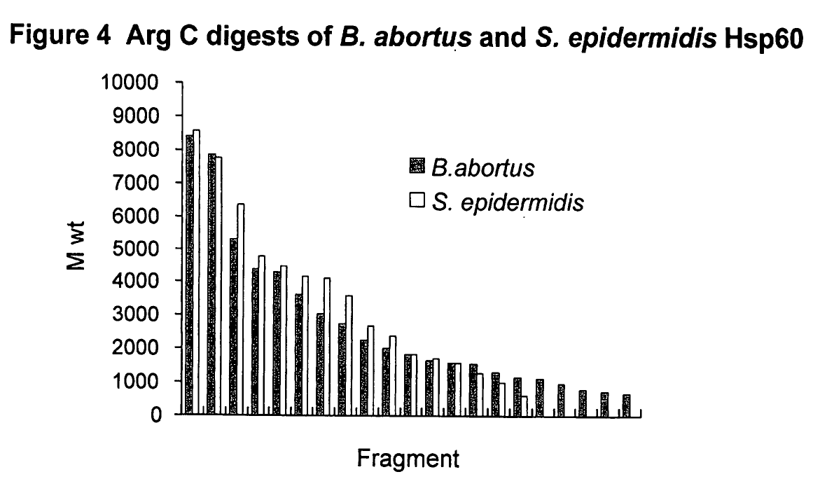

FIG. 4 is a graphical comparison of the peptide fingerprints resulting from Arg-C digests of HSp60 proteins of Brucella abortus and Staphylococcus epidennidis.

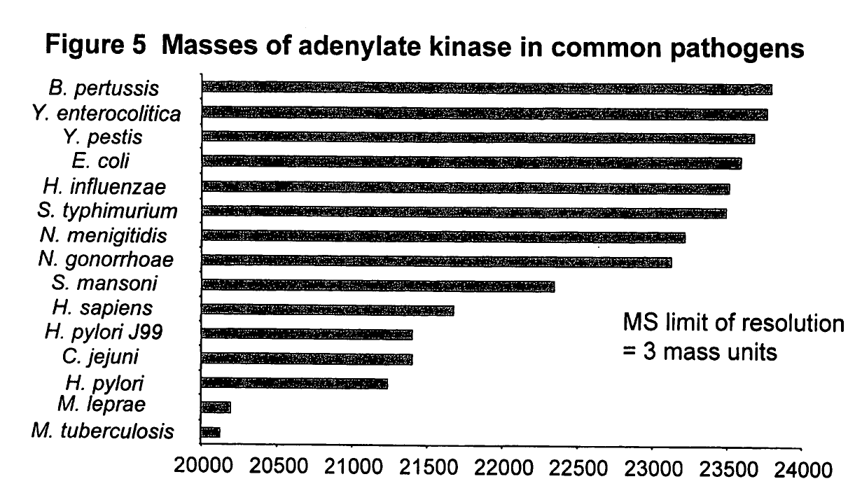

FIG. 5 is a graphical comparison of the molecular masses of adenylate kinase from a range of potentially pathogenic micro-organisms compared with human Hsp60.

### EXAMPLE 1

### An automatic Sampling and Identification System

With reference to FIG. 1, a vacuum device (not shown) is used to capture a sample of an aerosol suspected to contain pathogenic bacteria. The aerosol mixed with a carrier liquid, and the suspension is fed into the system (1) via a sampler (2). From there, the suspension is delivered to an ultrasonicator (4) within which ultrasound is used to break down the cell walls of any bacteria within the suspension, thereby releasing bacterial constituent proteins into a lysate. Inevitably, the lysate will also contain debris, so downstream from the ultrasonicator (4) is a filter (5), which prevents the passage of unwanted matter. In some cases, lysis may be improved by the use of a detergent, although this should not interfere with the immunoaffinity step downstream. Suitable mild non-ionic detergents are well-known in the art and include polyoxyethylene based detergents (such as Triton X-100 and X-114, Nonidet P40, and the Brij series) and n-octyl α-D-glucopyranoside.

Next comes an immunoaffinity module (6) in which one or more bacterial biomarkers, if present in the suspension, are isolated. Within the module (6) are one or more immobilised antibodies, specific for said biomarker(s). Biomarkers in the lysate passing through are thereby bound, whilst the remaining fluid passes through and is discarded. This step not only isolates the relevant biomarkers, but effectively concentrates them from what may be a very dilute lysate. After washing through the lysate, a small volume of elution buffer is admitted to the unit, to remove bound biomarkers.

The released biomarkers are delivered to a de-salter (8) whereupon they are de-salted before passing to an ion trap mass spectrometer (10) in which their individual molecular masses are determined. The combination of molecular masses obtained is then cross-referenced with a database of the molecular masses of the relevant biomarkers in a range of bacteria so as to identify any match. The output may be a specific identification, or the operator may simply be notified that the area is either “safe” or that it is “un-safe” and that appropriate protective measures are required.

In the event that biomarker proteins proteins are too large for individual analysis, eluted proteins may be sent to the de-salter (8) via an enzymatic digester (12) in which the proteins are cleaved at predictable points in their amino acid sequence and the resultant peptides analysed. The pattern of peptide molecular weights produced is diagnostic when compared to a database of such predicted peptides (see Example 3)

### EXAMPLE 2

### The use of Hsp60 as a Biomarker to Identify Potentially Pathogenic Bacteria

The average molecular mass of Hsp60 from a wide variety of organisms may be both predicted to a high degree of accuracy from the known amino acid sequence (corrected for mixture of isotopes present) and directly measured using the appropriate purified recombinant protein. Although Hsp60 is highly conserved across many species, not just bacteria, mass spectrometry allows highly accurate determination of mass and allows proteins molecules differing by as little as three mass units to be distinguished. Comparison of such measured values with a database of known values allows identification of the species involved, as shown in Table 1.

TABLE 1

| Hsp60 Mᵣ | Bacterium |
|---|---|
| 58015.3 Da | Chlamydia trachomatis |
| 57301.7 Da | Francisella tularensis |
| 57154.8 Da | Salmonella typhimurium |
| 56757.3 Da | Burkholderia pseudomallei |

FIG. 2 shows a graphical comparison the Hsp60 masses of a wider range of organisms illustrating that many species may be identified purely on the basis of their Hsp60 mass, as measured by mass spectrometry

However, In order to reduce the background of other proteins, some of which might have confusingly similar masses, affinity purification of a relevant biomarker is preferred. In the case of Hsp60, it is possible to immunoaffinity purify protein from cell lysates by means of cross-reacting antibodies. As an example, monoclonal antibody A57-E4 (Affinity Bioreagents Inc) binds to the linear epitope RGIDKA present in the Hsp60 of many potentially pathological organisms, including Bordetella pertussis, Burkholderia cepacia, Burkholderia pseudomallei, Chlamydia trachomatis, Chlamydophila pneumoniae, Chlamydophila psittaci, Coxiella bumetii, Haemophilus influenzae, Escherichia coli, Francisella tularensis, Klebsiella pneumoniae, Legionella pneumophila, Neisseria meningitidis, Pseudomonas aeruginosa, Salmonella typhi, Vibrio cholerae, Yersinia enterocolitica.

The use of such an antibody would therefore allow purification of Hsp60 from, and identification of, a wide range of potential pathogens. The binding of this antibody to the Hsp60 proteins of Francisella tularensis and Burkholderia pseudomallei was confirmed and quantified by a standard colorimetric indirect ELISA as shown in FIG. 3.

TABLE 2
Tryptic peptide map of Hsp60 from C trachomatis

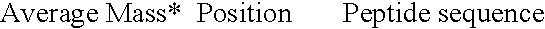

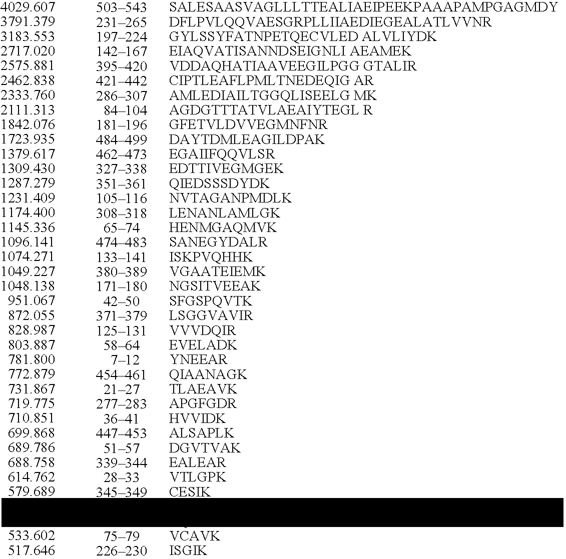

Peptides of mass <500 excluded.
*Average isotopically corrected mass (M = H)

TABLE 3
Tryptic peptide map of Hsp60 from C pneumoniae

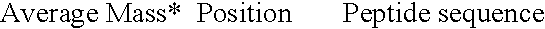

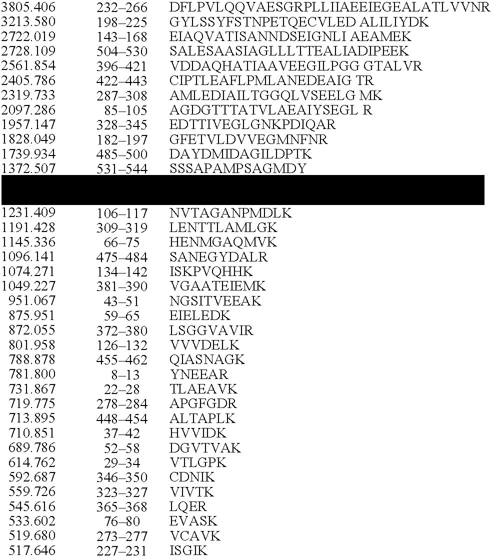

Peptides of mass <500 excluded.
*Average isotopically corrected mass (M + H)

### EXAMPLE 3

### Hsp60 Peptide Maps Derived from Hsp60 by Trypsin Digestion

As illustrated in Tables 2 and 3 above, enzymatic digestion of closely related Hsp60 proteins of similar overall molecular mass yields distinctive patterns of peptides that may be resolved by mass spectrometry. Trypsin cleaves peptides at the carboxy-peptide link of arginine and lysine residues (except where the next residue is a proline). Allowing for a mass accuracy of 0.01%, a few peptides are too similar to distinguish (boxed). In other cases, some very short peptides share identical composition and so have identical masses, and single free amino acids result from the cleavages. Even allowing for this, each peptide set constitutes a unique fingerprint, diagnostic of a specific organism from which the protein is derived.

### EXAMPLE 4

### Comparison of Arg-C Hsp60 peptides from Brucella Abortus and Staphylococcus Epidernidis

The endopeptidase Arg-C (clostripain), as its name suggests, cleaves the carboxy-peptide bonds of arginine. FIG. 4 shows a graphical comparison of the peptide fingerprints obtained from Arg-C digestion of Hsp60 from B. abortus and S. epidermidis. As shown in FIG. 3, the masses of the whole Hsp60 proteins from these organisms are similar (57649 and 57529, respectively including N-terminal methionines). However, the peptide sets obtained are quite distinct and characteristic of the organisms involved .

### EXAMPLE 5

### Use of Adenylate Kinase as a Diagnostic Biomarker

FIG. 5 shows a comparison of the masses of the highly conserved intracellular enzyme adenylate kinase from a variety of micro-organisms (bacteria and the protozoal parasite Shistosoma mansoni) as well as the human protein. Adenylate kinase is a nucleoside monophosphate kinase that catalyses the reversible phosphotransferase reactions between adenosine monophosphate, diphosphate and triphosphate. This enzyme plays an important role in the synthesis of nucleotides that are required for a variety of cellular metabolic processes, as well as for RNA and DNA synthesis. Adenylate kinase fulfills the criteria of a useful biomarker for the disclosed invention, in that it is highly conserved across species and yet each species has a unique protein distinguishable by mass. It is also consistently expressed and essential for metabolism.

### EXAMPLE 6

### Identification of E coli from Molecular Mass Measurement of Whole Hsp60 Biomarker by Electrospray Mass Spectrometry

To demonstrate the practicality of the invention, an anti-Hsp60 immunoaffinity column together with electrospray mass spectrometry were used to identify a bacterium, as follows.

### Methods

Preparation of Antibody Columns

The ligand (monoclonal antibody A57-E4 (Affinity Bioreagents Inc) was dialysed into 0.2M NaHCO₃, 0.5M NaCl, pH8.3 (coupling buffer) before binding to the column. The optimal volume was 1 ml with an optimal concentration of between 1 and 10 mg/ml.

A 1 ml NHS-activated Sepharose 4 in a Fast Flow Hi-Trap column (Pharmacia Biotech) was used. The column was washed with 3×2ml volumes of 1 mM HCl to remove the storage solution (isopropanol), keeping the flow rate to below a drop every two seconds to avoid compressing the matrix. The column was injected with ligand solution and incubated at room temperature for 30 minutes. The column was washed and deactivated by alternate washes with 0.5M ethanolamine, 0.5M NaCl, pH8.3 (buffer A) and 0.1M acetate, 0.5M NaCl, pH4.0 (buffer B) (3×2 Ml of buffer A, 3×2 ml of buffer B followed by 3×2 ml buffer A). The column was then equilibrated and stored in phosphate buffer containing 0.1% (w/v) sodium azide.

Sample Purification Method

All samples were run on NHS-activated columns on the AKTA prime system (Pharmacia Biotech). Samples were loaded in 20 mM sodium phosphate, pH7.5 and eluted in 3M urea at either pH8.0 or pH2.0. A sample volume of 1 ml was loaded onto the column via the sample loop and impurities were washed away with 5 ml of the phosphate buffer 6 ml of elution buffer was sent through the column, the first 1 ml of buffer was allowed to flow to waste while the following 2 mls were collected for analysis by electrospray mass spectroscopy. The column was regenerated by washing with 4 ml phosphate buffer, followed by 10 ml 3M urea and then 10 ml phosphate buffer in preparation for the next sample. The flow rate for all steps was 1 ml/min.

Mass Spectrometry Experimental Procedure

Eluent from the immunoaffinity column was collected as 2 ml fractions and stored overnight at 4° C. Samples were injected into a 2 ml holding loop. The contents of the holding loop were then loaded on to a C8 cartridge (Hichrom) at a flow of 1 ml/min in 20%B (A=0.1% TFA in water B=0.1% TFA in acetonitrile/water 90/10 (v/v)). After washing to remove buffer salts the protein was eluted into the Quattro II tandem quadrupole mass spectrometer (Micromass UK Ltd) using 90%B at a flow of 25 μl/min. Acquisition was performed in continuum mode. Scan range m/z 700-2000 at 5s per scan. Capillary voltage was 3 kV and cone voltage was ramped from 33V to 74V over the m/z range scanned. Source temperature was 80° C. and both LM Res and HM Res were set to 15.5. The elution peak from the cartridge was approximately 1 min in duration. The instrument was calibrated using horse heart myoglobin.

### Results

### Cross-reactivity of Antibody to Hsp60 Biomarkers

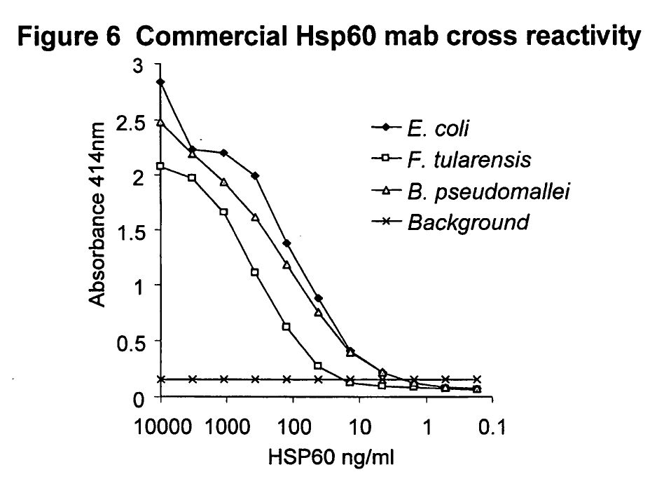

As shown in FIG. 6, a standard binding assay demonstrates that Hsp60 from a number of bacterial species cross-react with a monoclonal antibody raised against Chlamydia trachomatis Hsp 60 and binding a conserved epitope (RGIDKA). The binding curves indicate that such an antibody is suitable for immunopurification of Hsp60 biomarkers from a range of bacterial species.

### Identification of E coli Hsp60 K12 by Molecular Mass

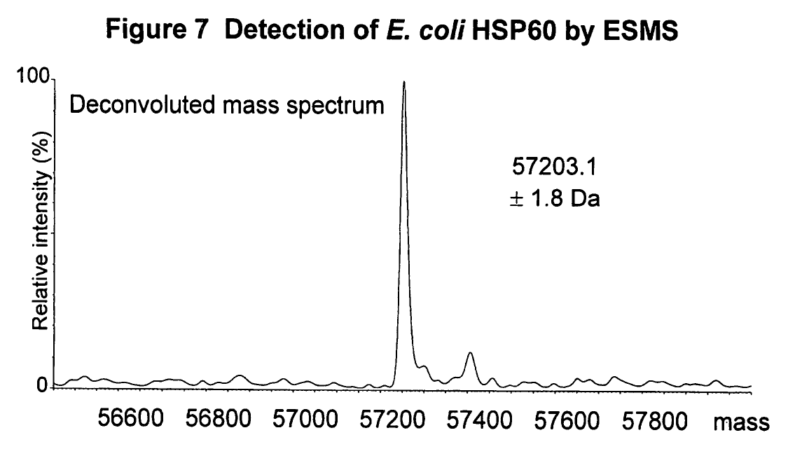

FIG. 7 shows the mass spectrum detected from eluate from the anti-Hsp60 immunoaffinity column that had been loaded with bacterial protein. The indicated molecular mass peak at 57203.1±1.8 Da matches that of Hsp 60 from an E.coli K12 variant reported by Burland et al (1995). The standard E coli Hsp60 mass, as detailed in the Swiss-Prot database entry P06130, is given as 57137 Da. However, the variant reported by Burland et al has two mutations, A261L and 1266M, which together give an expected mass of 57197. This is within the known mass accuracy of the instrument (±0.01%) and allows an unambiguous identification of not just the organism, but an individual strain and/or mutant.

### REFERENCES

Bakhtier R and Tse F L. Biological Mass Spectrometry: a Primer. Mutagenesis 15: 415-430 (2000).

Bonenberger T E, lhrke P J, Naydan D K and Affolter V K. Rapid identification of tissue micro-organisms in skin biopsy specimens from domestic animals using polyclonal BCG antibody. Vet Dermatol 12: 41-47 (2001)

Bundy J L and Fenselau C. Lectin and carbohydrate affinity capture surfaces for mass spectroscopic analysis of microorganisms. Anal Chem 73:751-757 (2001).

Burland V D, Plunkett G, Sofia H J, Daniels D L and Blattner F R. Analysis of the Escherichia coli genome VI: DNA sequence of the region from 92.8 through 100 minutes. Nucleic Acids Res 23: 2105-2119 (1995)

Chong B E, Wall D B, Lubman D M and Flynn S J. Rapid profiling of E. coli proteins up to 500 kDa from whole cell lysates using matrix-assisted laser desorption/ionization time-of-flight mass spectrometry. Rapid Commun Mass Spectrom 11: 1900-1908 (1997).

Downard K M. Contributions of mass spectrometry to structural immunology. J Mass Spectrom 35: 493-503 (2000).

Henderson S C, Valentine S J, Counterman A E and Clemmer D E. ESI/ion mobility/time-of-flight mass spectrometry for rapid and sensitive analysis of biomarker mixtures. Anal Chem 71:291-301 (1999).

Hoffman D, Hesselberth J and Ellington A S. Switching nucleic acids for antibodies. Nature Biotechnol 19: 313-314 (2001).

Krishnamurthy T, David M T, Stahl D C and Lee T D. Liquid chromatography/microspray mass spectrometry for bacterial investigations. Rapid Commun Mass Spectrom 13: 39-49 (1999).

Lacey J M, Bergen H R, Magera M J, Naylor S and O'Brien J F. Rapid determination of transferrin isoforms by immunoaffinity liquid chromatography and electrospray mass spectrometry. Clin Chem 47: 513-518 (2001).

Rowley A, Choudhary J S, Marzioch M, Ward M A, Weir M, Solari RCE and Blackstock W P. Applications of protein mass spectrometry in cell biology. Methods 20: 383-397 (2000).

Sigler P B, Xu Z, Rye H S, Burston S G, Fenton W A and Horwich A L. Structure and function in GroEL-mediated protein folding. Annu Rev Biochem 67: 581-608 (1998).

Willeke K and Baron P A. Aerosol Measurement—Principles, Techniques and Applications. Van Nostrand Reinhold (1993).

Yamashita T, Kiyoki E, Tomita Y and Taira H. Immunoaffinity purification and identification of the molecular chaperone calnexin. Biosci Biotechnol Biochem 63:1491-1493 (1999).

Yu L, Gaskell S J and Brookman J L. Epitope mapping of monoclonal antibodies by mass spectrometry: identification of protein antigens in complex biological systems. J Am Soc Mass Spectrom 9: 208-215 (1998).

1. A biomarker wherein species homologues of said biomarker derived from the majority of species in at least two genera of micro-organisms are substantially structurally similar, such that said structural similarity allows isolation of said biomarkers from different species of micro-organism and that each biomarker derived from each species of micro-organism in said genera has a unique molecular mass:

2. The biomarker of claim 1 wherein said biomarker is a protein and in that said structural similarity consists of substantial similarity of amino acid sequence.

3. The biomarker of claim 1 wherein said microorganisms are bacteria.

4. The biomarker of claim 1 wherein at least three species homologues share at least one common epitope allowing isolation by immunoaffinity chromatography.

5. The biomarker of claim 1 wherein said biomaiker is a heat shock protein.

6. The biomarker of claim 6 wherein said biomarker is Hsp60.

7. The biomarker of claim 1 wherein said biomarker is adenylate kinase.

8. A method of identifying micro-organisms comprising:
a. Identifying a biomarker wherein species homologues of said biomarker derived from the majority of species in at least two genera of micro-organisms are substantially structurally similar, such that said structural similarity allows isolation of said biomarkers from different species of micro-organism and that each biomarker derived from each species of micro-organism in said genera has a unique molecular mass;
b. Isolating said biomarkers by affinity chromatography directed towards regions of structural similarity;
c. Measuring the mass of said biomarkers by mass spectrometry; or
d. Analysing the combination of molecular mass data obtained with reference to a database and thereby deducing the species of microorganism present.

9. A method of identifying micro-organisms according to claim 8 wherein said biomarkers are isolated from a cell lysate.

10. The method of claim 8 wherein said biomarkers are isolated by means of immunoaffinity chromatography.

11. The method of claim 10 wherein immobilised antibodies bind specifically to cross-reacting epitopes present on marker molecules derived from a variety of micro-organism species.

12. The method of claim 8 wherein the additional step of cleaving the isolated biomarkers into defined fragments before determining their molecular mass by means of mass spectroscopy.

13. The method of claim 12 wherein said cleavage of said biomarkers is achieved by means of enzymatic digestion.

14. The method of claim 1 wherein the measurement of molecular mass of biomarkers or fragments thereof is by means of ion trap mass spectrometry.

15. A method of identifying macromolecular toxins comprising:
a. Isolating one or more toxins by affinity chromatography;
b. Measuring the molecular mass of said toxin(s) by means of mass spectrometry; or
c. Analysing the combination of molecular mass data obtained with reference to a database and thereby deducing the identity of the toxin(s) present.

16. An apparatus for the automatic performance of the method of claim 1 comprising:
a. A means for isolating said biomarkers by affinity chromatography;
b. A unit arranged for receiving and analysing said isolated biomarkers comprising a mass spectrometer operable to determine the molecular masses of said biomarkers; or
c. A data processing device arranged to receive data obtained from said mass spectrometer and to compare them with a database of known molecular masses and to thereby deduce the identity of the micro-organism detected.

17. The apparatus of claim 16 wherein said apparatus comprises a further unit comprising one or more immobilised proteolytic enzymes and arranged to receive biomarkers and to cleave them into peptides for analysis by mass spectrometry.


SEQUENCE LISTING (76 sequences)

SEQ ID NO: 1 - protein, 41 aa, Chlamydia trachomatis
SALESAASVAGLLLTTEALIAEIPEEKPAAAPAMPGAGMDY

SEQ ID NO: 2 - protein, 35 aa, Chlamydia trachomatis
DFLPVLQQVAESGRPLLIIAEDIEGEALATLVVNR

SEQ ID NO: 3 - protein, 28 aa, Chlamydia trachomatis
GYLSSYFATNPETQECVLEDALVLIYDK

SEQ ID NO: 4 - protein, 26 aa, Chlamydia trachomatis
EIAQVATISANNDAEIGNLIAEAMEK

SEQ ID NO: 5 - protein, 26 aa, Chlamydia trachomatis
VDDAQHATIAAVEEGILPGGGTALIR

SEQ ID NO: 6 - protein, 22 aa, Chlamydia trachomatis
CIPTLEAFLPMLTNEDEQIGAR

SEQ ID NO: 7 - protein, 22 aa, Chlamydia trachomatis
AMLEDIAILTGGQLISEELGMK

SEQ ID NO: 8 - protein, 21 aa, Chlamydia trachomatis
AGDGTTTATVLAEAIYTEGLR

SEQ ID NO: 9 - protein, 16 aa, Chlamydia trachomatis
GFETVLDIVEGMNFNR

SEQ ID NO: 10 - protein, 16 aa, Chlamydia trachomatis
DAYTDMLEAGILDPAK

SEQ ID NO: 11 - protein, 12 aa, Chlamydia trachomatis
EGAIIFQQVMSR

SEQ ID NO: 12 - protein, 12 aa, Chlamydia trachomatis
EDTTIVEGMGEK

SEQ ID NO: 13 - protein, 11 aa, Chlamydia trachomatis
QIEDSSSDYDK

SEQ ID NO: 14 - protein, 12 aa, Chlamydia trachomatis
NVTAGANPMDLK

SEQ ID NO: 15 - protein, 11 aa, Chlamydia trachomatis
LENANLAMLGK

SEQ ID NO: 16 - protein, 10 aa, Chlamydia trachomatis
HENMGAQMVK

SEQ ID NO: 17 - protein, 10 aa, Chlamydia trachomatis
SANEGYDALR

SEQ ID NO: 18 - protein, 9 aa, Chlamydia trachomatis
ISKPVQHHK

SEQ ID NO: 19 - protein, 10 aa, Chlamydia trachomatis
VGAATEIEMK

SEQ ID NO: 20 - protein, 10 aa, Chlamydia trachomatis
NGSITVEEAK

SEQ ID NO: 21 - protein, 9 aa, Chlamydia trachomatis
SFGSPQVTK

SEQ ID NO: 22 - protein, 9 aa, Chlamydia trachomatis
LSGGVAVIR

SEQ ID NO: 23 - protein, 7 aa, Chlamydia trachomatis
VVVDQIR

SEQ ID NO: 24 - protein, 7 aa, Chlamydia trachomatis
EVELADK

SEQ ID NO: 25 - protein, 6 aa, Chlamydia trachomatis
YNEEAR

SEQ ID NO: 26 - protein, 8 aa, Chlamydia trachomatis
QIAANAGK

SEQ ID NO: 27 - protein, 7 aa, Chlamydia trachomatis
TLAEAVK

SEQ ID NO: 28 - protein, 7 aa, Chlamydia trachomatis
APGFGDR

SEQ ID NO: 29 - protein, 6 aa, Chlamydia trachomatis
HVVIDK

SEQ ID NO: 30 - protein, 7 aa, Chlamydia trachomatis
ALSAPLK

SEQ ID NO: 31 - protein, 7 aa, Chlamydia trachomatis
DGVTVAK

SEQ ID NO: 32 - protein, 6 aa, Chlamydia trachomatis
EALEAR

SEQ ID NO: 33 - protein, 6 aa, Chlamydia trachomatis
VTLGPK

SEQ ID NO: 34 - protein, 5 aa, Chlamydia trachomatis
CESIK

SEQ ID NO: 35 - protein, 5 aa, Chlamydia trachomatis
VIVSK

SEQ ID NO: 36 - protein, 4 aa, Chlamydia trachomatis
LQER

SEQ ID NO: 37 - protein, 5 aa, Chlamydia trachomatis
VCAVK

SEQ ID NO: 38 - protein, 5 aa, Chlamydia trachomatis
ISGIK

SEQ ID NO: 39 - protein, 35 aa, Chlamydophila pneumoniae
DFLPVLQQVAESGRPLLIIAEEIEGEALATLVVNR

SEQ ID NO: 40 - protein, 28 aa, Chlamydophila pneumoniae
GYLSSYFSTNPETQECVLEDALILIYDK

SEQ ID NO: 41 - protein, 26 aa, Chlamydophila pneumoniae
EIAQVATISANNDSEIGNLIAEAMEK

SEQ ID NO: 42 - protein, 27 aa, Chlamydophila pneumoniae
SALESAASIAGLLLTTEALIADIPEEK

SEQ ID NO: 43 - protein, 26 aa, Chlamydophila pneumoniae
VDDAQHATIAAVEEGILPGGGTALVR

SEQ ID NO: 44 - protein, 22 aa, Chlamydophila pneumoniae
CIPTLEAFLPMLANEDEAIGTR

SEQ ID NO: 45 - protein, 22 aa, Chlamydophila pneumoniae
AMLEDIAILTGGQLVSEELGMK

SEQ ID NO: 46 - protein, 21 aa, Chlamydophila pneumoniae
AGDGTTTATVLAEAIYSEGLR

SEQ ID NO: 47 - protein, 18 aa, Chlamydophila pneumoniae
EDTTIVEGLGNKPDIQAR

SEQ ID NO: 48 - protein, 16 aa, Chlamydophila pneumoniae
GFETVLDVVEGMNFNR

SEQ ID NO: 49 - protein, 16 aa, Chlamydophila pneumoniae
DAYTDMIDAGILDPTK

SEQ ID NO: 50 - protein, 14 aa, Chlamydophila pneumoniae
SSSAPAMPSAGMDY

SEQ ID NO: 51 - protein, 12 aa, Chlamydophila pneumoniae
EGAIICQQVLAR

SEQ ID NO: 52 - protein, 11 aa, Chlamydophila pneumoniae
QIEDSTSDYDK

SEQ ID NO: 53 - protein, 12 aa, Chlamydophila pneumoniae
NVTAGANPMDLK

SEQ ID NO: 54 - protein, 11 aa, Chlamydophila pneumoniae
LENTTLAMLGK

SEQ ID NO: 55 - protein, 10 aa, Chlamydophila pneumoniae
HENMGAQMVK

SEQ ID NO: 56 - protein, 10 aa, Chlamydophila pneumoniae
SANEGYDALR

SEQ ID NO: 57 - protein, 9 aa, Chlamydophila pneumoniae
ISKPVQHHK

SEQ ID NO: 58 - protein, 10 aa, Chlamydophila pneumoniae
VGAATEIEMK

SEQ ID NO: 59 - protein, 10 aa, Chlamydophila pneumoniae
NGSITVEEAK

SEQ ID NO: 60 - protein, 7 aa, Chlamydophila pneumoniae
EIELEDK

SEQ ID NO: 61 - protein, 9 aa, Chlamydophila pneumoniae
LSGGVAVIR

SEQ ID NO: 62 - protein, 7 aa, Chlamydophila pneumoniae
VVVDELK

SEQ ID NO: 63 - protein, 8 aa, Chlamydophila pneumoniae
QIASNAGK

SEQ ID NO: 64 - protein, 6 aa, Chlamydophila pneumoniae
YNEEAR

SEQ ID NO: 65 - protein, 7 aa, Chlamydophila pneumoniae
TLAEAVK

SEQ ID NO: 66 - protein, 7 aa, Chlamydophila pneumoniae
APGFGDR

SEQ ID NO: 67 - protein, 7 aa, Chlamydophila pneumoniae
ALTAPLK

SEQ ID NO: 68 - protein, 6 aa, Chlamydophila pneumoniae
HVVIDK

SEQ ID NO: 69 - protein, 7 aa, Chlamydophila pneumoniae
DGVTVAK

SEQ ID NO: 70 - protein, 6 aa, Chlamydophila pneumoniae
VTLGPK

SEQ ID NO: 71 - protein, 5 aa, Chlamydophila pneumoniae
CDNIK

SEQ ID NO: 72 - protein, 5 aa, Chlamydophila pneumoniae
VIVTK

SEQ ID NO: 73 - protein, 4 aa, Chlamydophila pneumoniae
LQER

SEQ ID NO: 74 - protein, 5 aa, Chlamydophila pneumoniae
EVASK

SEQ ID NO: 75 - protein, 5 aa, Chlamydophila pneumoniae
VCAVK

SEQ ID NO: 76 - protein, 5 aa, Chlamydophila pneumoniae
ISGIK